(19) United States
(12) Patent Application Publication
(10) Pub. No.: US 2009/0149776 A1
(43) Pub. Date: Jun. 11, 2009

(54) OPTICAL SENSOR FOR DETECTING INFECTION AND OTHER ANOMALOUS CONDITIONS ASSOCIATED WITH CATHETER SYSTEMS

(76) Inventor: Scott C. Adams, Placentia, CA (US)

Correspondence Address: LOZA & LOZA LLP, 305 N. Second Avenue, #127, Upland, CA, 91786-6064, US

(21) Appl. No.: 12/328,750

(22) Filed: Dec. 4, 2008

Related U.S. Application Data
(60) Provisional application No. 60/992,677, filed on Dec. 5, 2007.

(51) Int. Cl. A61B 5/00

(52) U.S. Cl. 600/584

(57) ABSTRACT

A device and method are provided to monitor for early detection of anomalous conditions in a discharge fluid through a catheter tube. A liquid sensor is configured to detect the presence of liquid in the catheter tube. A unique photo-Darlington turbidity sensor may be configured to monitor turbidity of a discharge liquid exiting through the catheter tube. A color sensor may be configured to monitor color of the discharge liquid exiting through the catheter tube. A processing circuit may be coupled to the liquid sensor, photo-Darlington turbidity sensor, and color sensor and configured to determine whether an anomalous condition exists based on readings provided by the liquid sensor, photo-Darlington turbidity sensor, and/or color sensor. A warning device provides an alert if an anomalous condition is detected.

### CLAIM OF PRIORITY UNDER 35 U.S.C. §119

The present Utility Application for Patent claims priority to U.S. Provisional Application No. 60/992,667 entitled “Optical Sensor for Detecting Infection and Other Anomalous Conditions Associated with Catheter Systems” filed Dec. 5, 2007, and hereby expressly incorporated by reference herein.

### FIELD

The present invention relates to an optical sensing/monitoring system for detecting anomalous conditions associated with catheter systems.

### BACKGROUND

Urinary (Foley) catheters are utilized worldwide by patients in both in- and out-patient environments. It is estimated that 25% of all in-patients admitted to hospitals in the United States will have at least one urinary catheter administered during their stay. As the total Hospital Admissions Rate is over 37 Million annually, approximately 10 million urinary catheter systems in the U.S. alone will be utilized annually. In the case of Peritoneal Dialysis (PD) Catheters, there are approximately 40,000 patients in the U.S. utilizing PD catheters at least 3 times (often more frequently) daily. This represents nearly 4 million units annually in the U.S. alone.

Thus, in the example of Foley and PD Catheters alone, there may be at least 14 million units used annually, and that does not include statistics from managed care (convalescent) and other out-patient environments. Additionally, statistics for Canada, Europe and Australia are similar and represent another 42 million units utilized annually. Overall, it is estimated that 56 million of these catheter systems are used worldwide annually.

It is not uncommon for patients using catheters to suffer complications, such as infections. For Foley type catheters, urinary/bladder infection dominates, while for PD catheters, peritonitis dominates. In either case, infection often leads to further complications and costs to the health care system. For PD patients alone, statistics indicate that it's not a question of “if” an infection will occur, but rather “when” it will occur. Furthermore, it is estimated that 6% of PD patients will die as a result of PD related peritonitis.

Constant or continuous visual monitoring of the catheter effluent is important in the early detection of potentially anomalous conditions. Unfortunately, achieving an effective, constant monitoring of the catheter system is difficult. For example, elderly outpatients with poor eyesight or non-ambulatory in-patients who are unable to examine their own catheter cannot effectively monitor such for the onset of anomalous conditions. Furthermore, in-patient monitoring currently requires periodic nursing intervention or examination, which in turn taxes the nurse workload.

Prior art monitoring systems have utilized other type of sensors which either lack the required sensitivity (unable to achieve adequate signal-to-noise ratio, SNR) and/or employ sensor schemes which do not lend themselves to low-power, disposable applications (e.g. PIN diodes and CCD sensors). These monitoring systems typically work by detecting particle sizes in the catheter outflow to differentiate from unwanted signals. For example, particle size is used to detect the presence of an infection by differentiating between red blood cells and white blood cells, each of which has different particle sizes. These monitoring systems utilize sensors, such as PIN diodes and charge-coupled device (CCD) sensors, to detect the particle sizes; however, these types of sensors lack the necessary sensitivity to achieve an adequate signal-to-noise ratio (SNR) to detect early onset infection and to effectively differentiate from false positives. Additionally, these monitoring system do not lend themselves to low-power, disposable applications as the use of CCD sensors result in relatively high-power consumption.

Consequently, a more sensitive, low power, constant and continuous monitoring mechanism is needed that can be used in any environment, portable and/or disposable, including inpatient and outpatient environments while providing sufficiently high resolution to detect the early onset of an anomalous condition, such as an infection, based on the optical analysis of the discharge fluid from a catheter.

### SUMMARY

One feature is aimed at the detection of infection and other anomalous conditions associated with Catheter Systems. By employing unique mechanisms to monitor the “Color” and “Clarity” of catheter effluent, and by comparison to “known normal” conditions of such, the device is designed to alert health care givers or ambulatory patients in the event that abnormal conditions develop or begin to develop (early detection), or, in the case of a pre-existing anomalous condition, to indicate if that condition is improving or not. The types of catheters in use which may benefit from the present invention include, but are not limited to, urinary (Foley type) and Peritoneal Dialysis catheters.

In one example, an apparatus is provided for detecting anomalous conditions associated with effluents of catheter systems. The apparatus may include a liquid sensor, a color sensor, and/or a clarity sensor. The (refractive) liquid sensor may sense the presence of the effluent in a catheter system. The color sensor may be adapted to sense a color of the effluent. The clarity sensor may be adapted to sense a clarity of the effluent. If the liquid sensor detects the presence of the effluent, color data collected by the color sensor and clarity data collected by the clarity sensor may be considered valid, otherwise such data may be ignored.

In one implementation, the liquid sensor may include a light source and a light sensor. The liquid sensor may detect a difference in displacement of the effluent by refraction when the effluent is present versus when it is absent.

The clarity sensor may include a sensitive light scattering detector to detect light scattered from a light source passing through the effluent at a right and oblique angle in order to determine the clarity of the effluent. The dynamic range of the clarity sensor may be between one (1) and five hundred (500) Nephelometric Turbidity Units (NTU) and a resolution sensitivity of at least five (5) NTU. The clarity sensor may include a photo Darlington detector that is adapted to achieve a desired absolute signal measurement and a desired signal-to-noise ratio sufficient to achieve the dynamic range and resolution sensitivity. The clarity sensor may further include a photo Darlington base drive feedback circuit to control the gain of the photo Darlington detector so as to eliminate non-linearity of gain associated with changes in ambient light and other noise sources. The clarity sensor may also include a photo Darlington base drive feedback circuit to adjust absolute gain of the Darlington detector to provide greater overall dynamic range of the clarity sensor. A low power laser diode may be utilized as the light source. In one example, the laser diode may be positioned at a right and oblique angle to the photo Darlington detector so as to minimize light reflection noise within a test cavity and sense scattered light resulting from the presence of turbidity in the effluent. A white light light-emitting diode may be utilized to provide a transmitting light source for determining color of the catheter effluent.

According to one feature, the sensed color and clarity of the effluent may be used to determine the occurrence of an anomalous condition. A diffuser may be utilized to provide a diffuse backlit light source for determining color of the catheter effluent.

A microprocessor may also be coupled to the liquid sensor, clarity sensor, and color sensor and adapted to collect effluent data and ascertain whether an anomalous condition is present. The microprocessor may be adapted to: (a) perform data trending analysis on collected effluent data, and (b) trigger an alarm if the collected effluent data indicates a certain threshold has been exceeded. The microprocessor may also be adapted to detect at least one of: (a) anomalous conditions associated with the effluent catheter systems, (b) early on-set of anomalous conditions, and/or (c) improvement of an already existing anomalous condition associated with catheter systems. The microprocessor may also be adapted to perform statistical analysis of collected temporal sensor data to distinguish true alarm events from transient or noise events. Such transient events may include bubbles and/or turbulence in the effluent and other transient conditions such as effluent fibrin, etc. The microprocessor may also be adapted to measure elapsed time between liquid effluent detections by the liquid sensor, and trigger an alarm if a time threshold between liquid detections is exceeded to indicate un-timely effluent production.

In some implementations, the apparatus may be removable or permanently attached to an effluent drain tube which is utilized as both an effluent flow path and a test cell for the sensors. Consequently, the effluent drain tube and apparatus may be disposable. In other implementations, the apparatus may be attached to an effluent drain bag which is utilized as both an effluent reservoir and a test cell for the sensors.

In other implementations, the apparatus may be integrated onto the effluent drain bag or tube assembly of a Foley urinary catheter system to detect the onset, existence, or the improvement from a condition of urinary tract infections, presence of blood, presence of infection or other anomalous condition associated with urinary catheters. In other examples, the apparatus may be integrated onto the effluent drain bag/tube assembly of any catheter system to detect the onset, existence, or the improvement from a condition of the presence of blood, presence of infection or other anomalous condition associated with that catheter system.

### BRIEF DESCRIPTION OF THE DRAWINGS

Various features of the present invention will be better understood from the following detailed description of an exemplary embodiment of the invention, taken in conjunction with the accompanying drawings in which like reference numerals refer to like parts.

FIG. 1 is a schematic representation of a catheter monitoring device for constantly and/or continuously monitoring one or more characteristics of discharge liquids from a catheter system.

FIG. 2 illustrates an example in which the monitoring device may be housed within a “clamshell” attachment so that the monitoring device may be enclosed around the drain tube of an associated catheter.

FIG. 3 is a block diagram illustrating the functional components of a monitoring device according to one example.

FIG. 4 is a block diagram illustrating the functional components of an example of a monitoring device that monitors effluent clarity, including a unique sensor and feedback arrangement that enables the present invention to accomplish the sensitivity and dynamic range goals necessary to successfully detect early onset of anomalous conditions associated with catheter systems.

FIG. 5 illustrates an example in which the monitoring device may be attached directly to the catheter collection bag, rather than a tube.

FIG. 6 illustrates a method for monitoring for anomalous conditions of an effluent of a catheter system.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention.

In the following description, certain terminology is used to describe certain features of one or more embodiments of the invention. The term “catheter system”, includes, but is not limited to, urinary catheters also known as Foley catheters, Peritoneal Dialysis (PD) Catheters, and/or any other types of catheters that may be used in the medical field and includes the effluent drain elements of the catheter systems such as the drain tubes and drain bags. The term “discharge liquids” or “effluent” refers to dialysate, urine, or any type of liquid and/or bodily fluid that may be discharged by a body of an individual via the catheter system.

### Overview

An effective way of dealing with infections and anomalous conditions associated with catheter systems (e.g., urinary/bladder infection, peritonitis, etc.) is early detection. The most common method employed (aside from periodic blood, dialysate, or urine testing) is by visual inspection of the liquid exiting the catheter. “Color” and “Clarity” of the urine or dialysate (depending on catheter type) is an excellent indicator of a potentially anomalous condition evolving. Typically, a “cloudy” fluid is indicative of the onset of infection. Color can also be indicative anomalous conditions such as a more advanced infection (e.g. green) or an even more serious condition (e.g. red indicating blood in the urine or dialysate).

One feature of the present invention provides an automated, no to low-maintenance monitoring device for constant/continuous inspection of catheter discharge liquids to detect infection and other anomalous conditions. That is, the monitoring device may include one or more sensors that sense one or more characteristics of the discharge liquids or effluent to determine whether an abnormal condition is present. These characteristics may include, but are not limited to “Color” and/or “Clarity” as discussed above. The sensed characteristics of the discharge liquid may be compared to “known normal” conditions of the discharge liquid so that when an abnormal condition develops or begins to develop, the monitoring device may alert health care givers or ambulatory patients of the abnormal condition. Alternatively, in the case of a pre-existing anomalous condition, the monitoring device may alert health care givers or ambulatory patients to whether or not the pre-existing anomalous condition is improving.

Another feature provides for an easy-to-install, replaceable, low power consumption, small-form factor monitoring device that may be attached around a catheter tube (or discharge bag) to sense changes in the fluid flowing therein. This allows for the monitoring device to be easily integrated into existing catheter drain systems providing each patient with an inexpensive in-situ monitoring capability. Alternatively, the monitoring device may be integrated into a catheter tube, discharge bag, support equipment, and/or a monitoring device.

Yet another feature provides a monitoring device that is capable of automatically detecting the presence of discharge liquid and then accurately measuring the color and clarity of the discharge liquid. By knowing when discharge liquid is present, the monitoring device can provide power management to reduce or minimize the power consumption of the device by causing the device to implement a low power sleep mode when liquid is not present, thus providing a low power, battery operated, monitoring device.

### Example Catheter Monitoring System

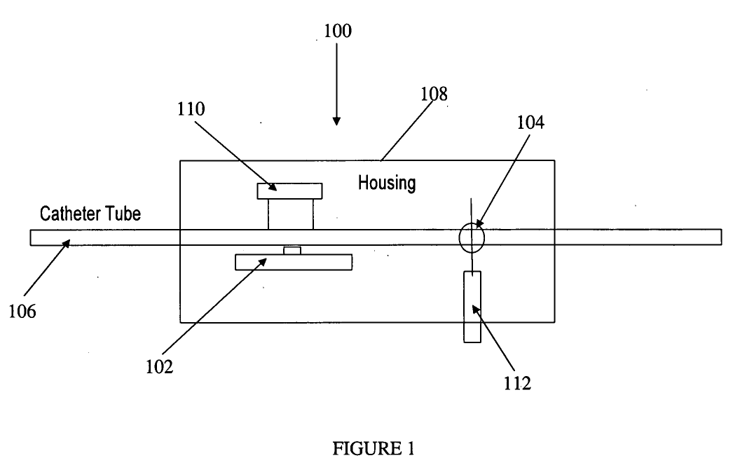

FIG. 1 is a schematic representation of a catheter monitoring device 100 for constantly and/or continuously monitoring one or more characteristics of discharge liquids from a catheter system, according to one embodiment. The monitoring device 100 may include a housing 108 having a first optical sensor 102 and a diffuse multi-spectral light source 110 for detecting/measuring, for example, the color of the discharge liquid exiting through the catheter tube 106 and a second optical sensor 104 for measuring the clarity/turbidity of the discharge liquid exiting through the catheter tube 106 by unique means of measuring the scattered light from a light source 112. Turbidity is the cloudiness or haziness of a fluid caused by individual particles (suspended solids) that are generally invisible to the naked eye and is typically measured as Nephelometric Turbidity Units (NTU). In this example, both the color and clarity of exiting catheter discharge liquids may be measured by the first and second optical sensors 102 and 104, respectively. By monitoring the exiting discharge liquid through the clear catheter tube, the interior surfaces of the catheter are isolated from other external sources of contamination, thus eliminating the possibility of introducing additional sources of infection.

The monitoring device 100 may be attached, either permanently or temporarily, to the catheter tube 106 of the catheter system, for example, by a clam-shell mechanism attached around the draining catheter tube 106 or attached to a drainage sack or discharge bag (not shown) of a catheter system in such a manner so as to facilitate “viewing” of the catheter discharge liquid by both sensors 102 and 104.

The sensors 102 and 104 may measure a characteristic of the discharge liquid, which is then analyzed by an on-board circuit (e.g., microprocessor) located within the housing 108. If an abnormal condition is sensed in the discharged liquid by the first and/or second sensors 102 and 104, a warning may be provided, such as an alarm, audible, visual or electronic (RF) indicator. For instance, if anomalous color and/or clarity are detected in the discharge liquid, such warning may be activated.

When the alarm is activated, it warns the patient or caretaker that there may be an infection or another anomalous or abnormal condition with a patient. As discussed above, the alarm may be audible, visual, and/or telemetry based, the exact configuration may be determined by the patient environment/characteristics (e.g. an ICU (Intensive Care Unit) environment might utilize telemetry or wireless monitoring, whereas out-patient utilization by an elderly patient with eyesight limitations might employ an audible alarm only).

To collect data from the discharge liquid or effluent, a catheter tube 106 of a catheter system may be inserted into, or positioned adjacent to, the housing 108 of the monitoring device 100 (as shown in FIG. 1).

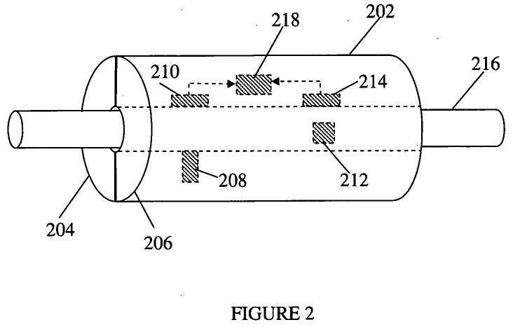

FIG. 2 illustrates an example in which the monitoring device may be housed within a “clamshell” attachment 202 (two-piece housing 204 and 206) so that the monitoring device may be enclosed around the drain tube 216 of an associated catheter. The first optical sensor 210 may be a color detector that detects color from the discharge liquid with a backlit source 208, such as a diffuse multi spectral light source.

The second optical sensor 214 may be a detector, with a field of view orthogonal to the laser path through the catheter tube 216 that detects light scattering from a collimated laser source 212 to determine the clarity/turbidity of the discharge liquid. In one example, the second optical sensor 214 may be a high sensitivity photo-Darlington turbidity sensor having a drive circuit and amplifying/filtering circuit for detecting turbidity levels. The drive circuit may be a feedback circuit which is used to control the gain of the photo-Darlington clarity/turbidity sensor so as to eliminate non-linearity of gain associated with changes in ambient light and other noise sources. Additionally, the drive circuit may be used to adjust absolute gain of the sensor to provide greater overall dynamic range of the sensor by tuning the overall gain of the sensor for any given circumstance, thus providing the ability to continuously select the gain for a wide range of signals. A microprocessor 218 or other circuit may control the operation of the sensors and trigger an alarm when an anomalous condition is detected.

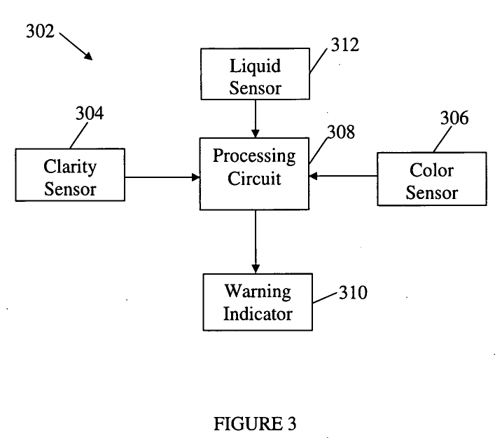

FIG. 3 is a block diagram illustrating the functional components of a monitoring device 302 according to one example. A first sensor may be a color sensor 306, such as a Red Green Blue (RGB) detector with a white backlight source, a second sensor may be a clarity sensor 304 (e.g., photo-Darlington detector) for detecting clarity or cloudiness within the catheter effluent and a third sensor may be a liquid sensor 312 for detecting the presence of liquid discharge. The first, second and third sensors 306, 304 and 312 are coupled to a processing circuit 308. The processing circuit 308 may be configured to operate (or control) and interpret data from the color, clarity and liquid sensors 306, 304 and 312 and provide a warning via a warning indicator 310 if an abnormal condition is detected by the sensors as the processing circuit 308 may control both visible and audible alarms if certain thresholds are exceeded by any sensor. The processing circuit 308 may use stored calibration and threshold alarm values to determine if a warning should be provided.

The liquid sensor 312 may be used to collect trending information, e.g., to determine how frequently liquid is being discharged through the catheter. Additionally, the monitoring device may be configured such that the color and/or clarity sensors 306 and 304 operate and/or collect data or measurements only when liquid is present or detected by the liquid sensor 312.

By using a color sensor 306, such as an RGB color detector, the effluent monitoring device 302 may detect the presence of blood or other anomalous conditions. An RGB color detector may be used to detect the presence of red blood cells (RBC) or other anomalous colors. This is preferable to other approaches (e.g., using CCD type sensors to differentiate between particle sizes of red blood cells and white blood cells) since the RGB color sensor 306 is low-power consumption and, consequently, suitable to disposable, battery operated applications.

The clarity sensor 304 may be implemented using a photo-Darlington sensor that has a sufficiently high signal-to-noise ratio to detect the onset of infections or abnormalities in the discharge liquid. Consequently, the clarity sensor 304 may be capable of sensing clarity or turbidity levels from 1 Nephelometric Turbidity Unit (NTU) to over 500 NTU with resolution as low as 5 NTU resolution, for example. This is typically not achievable with PIN diode or CCD array configurations. However, photo-Darlington detectors may be non-linear to light intensity, which makes them undesirable and/or difficult to use for these types of implementations.

### Photo-Darlington Based Sensor

In the design described above, a photo-darlington detector may be utilized with a unique drive circuit designed to detect turbidity levels ranging from 1 NTU to 500 NTU, with resolution as low as 1-5 NTU. Prior art detectors lack the sensitivity to achieve that range and resolution. The use of a photo-darlington in this application is unique in that such detectors are not usually employed in this type of application due to certain disadvantages. The primary disadvantage of photo-darlington sensors is their gain dependence with light intensity, thus resulting in non-linear behavior and an inherent susceptibility to ambient light levels. Modulation and post filtering of the signal alone is unable to eliminate signal contribution due to these changes in gain with ambient light.

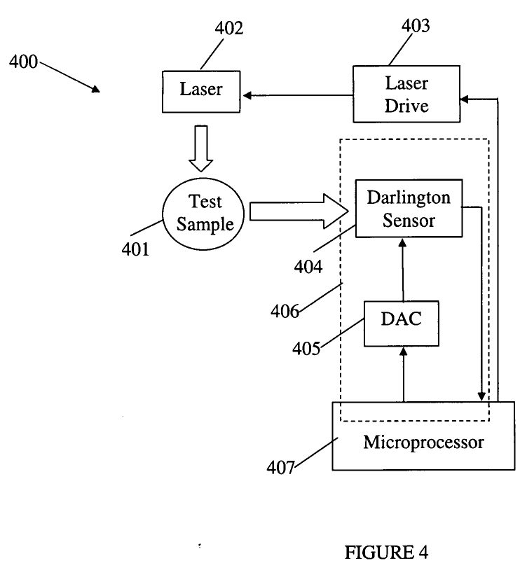

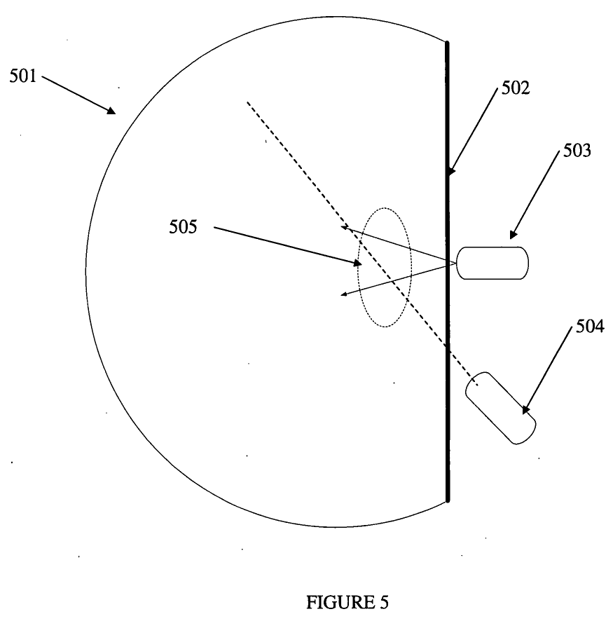

FIG. 4 is a block diagram illustrating the functional components of an example of a monitoring device 400 that monitors effluent clarity, including a unique sensor and feedback arrangement that enables the present invention to accomplish the sensitivity and dynamic range goals necessary to successfully detect early onset of anomalous conditions associated with catheter systems. This design eliminates the problems and disadvantages noted above by providing microprocessor controlled, real time feedback to the darlington base compensating for changes in ambient light levels.

A feedback methodology is illustrated in block 406. An additional advantage of such a closed loop feedback design is that the overall gain of the sensor can be tuned for any given circumstance, thus providing the ability to continuously select gain for a wide range of signals, hence the enhanced dynamic range of the detector. The result is a detector system which has the high gain advantage of a photo-darlington detector (typically 10,000 times greater than PIN diodes, for example), without the induced noise associated with the photo-darlington's non-linear gain dependence with overall light levels, including ambient light noise. The resulting SNR is such that the detector is capable of sensing turbidity levels from 1 NTU to over 500 NTU with as great as 1-5 NTU resolution while still utilizing a low power, eye safe laser diode as the scattering source. In applications such as PD catheter monitoring, bench test data indicates that this level of sensitivity and resolution is required in order to differentiate between “normal” effluent and “early onset” infection, thus the present invention is uniquely capable of detecting early onset infection for these types of catheter systems.

Ambient light levels are measured with a sensor 404 and a corresponding collector-emitter current is derived for any given light condition. This measurement is fed back to a microprocessor 407 which in turn generates an injected base current via a Digital to Analog Converter (DAC) 405 to “stabilize” or bring back the collector-emitter current to some pre-set, pre-defined level, thus compensating for any given ambient light level.

FIG. 7 illustrates an example of the Photo-Darlington Feedback methodology in greater detail. The collector-emitter current is monitored by the microprocessor 702 by ADC (Analog to Digital) Conversion at the collector as shown at 701 by measuring the voltage at that point. The measurement at 701 is taken at a time that corresponds to a sensor measurement of the ambient light level only (laser diode off). The measurement at 701 is compared to a pre-set, pre-defined voltage setting determined initially during device calibration and a corresponding base current is injected at 703 so as to bring the collector voltage at 701 back to the pre-defined voltage setting. This effectively adjusts gain for any given ambient light condition. Additionally, the pre-set, pre-defined collector voltage at 701 can be varied to produce a different overall operating gain, thus facilitating a larger dynamic range of sensitivities for different levels of detection.

In one example, the monitoring device 400 may also include a third sensor, such as a refractive liquid sensor, for detecting the presence of effluent. The third sensor may include a light source (such as a light emitting diode (LED)) and a sensor that detects the difference in displacement of the LED image by refraction when liquid is present versus when it is not present.

As optical measurements of the catheter discharge liquid are performed in the discharge liquid “flow path”, the liquid sensor verifies the presence of continuous liquid in the flow path, over the measurement time period to ensure accurate measurements. In addition to detecting the presence of liquid, the third sensor may also provide power management to reduce or minimize the power consumption of the device by causing the device to implement a low power sleep mode when liquid is not present, thus providing a low power, battery operated, monitoring device.

By utilizing a liquid sensor, “dry times”, i.e. time elapsed between liquid events, may be measured. By knowing the time elapsed between liquid events, it may be determined that liquid is being timely produced. For example, with urinary Foley catheters, it may be important to know that urine is being produced by the body which is a critical observation for overall kidney and bladder function. Furthermore, the ability to detect and verify the presence of discharge liquid may also be important when other types of catheters are used as excessive “dry times” may also be characterized as an “anomalous” condition associated with catheter systems. Additionally, the monitoring system may be configured such that the color and/or turbidity sensors operate and/or collect data or measurements only when liquid is present or detected by the liquid sensor.

In one embodiment, different types of alarms may be used to indicate different anomalies. For example, there may be a first audible sound to indicate an infection and there may be a second audible sound to indicate a different anomaly.

It should be noted that although the monitoring device as illustrated in FIGS. 1 and 2 may be attached to the catheter tube (above a collection bladder), this is by way of example only and the monitoring device may be attached to other parts of the catheter system. For instance, FIG. 5 illustrates an example in which the monitoring device may be attached directly to the catheter collection bag, rather than a tube. A rigid window 502 would facilitate the mounting of both a light source 504 and detector assembly 503 so as to facilitate a set viewing region 505 with a known distance from the detector(s) and residing within the liquid contained within the collection bag 501.

Referring again to FIG. 4, the processing circuit or microprocessor 407 may be configured to control different modes of operation of the monitoring device, such as a sleep, absolute measurement, and trending measurement modes, as well as analyze data collected from the sensors and distinguish anomalous conditions from transient noise events including liquid transients, such as bubbles and turbulence, and other transients, such as fibrin.

In the Sleep mode of operation, the microprocessor may be used to “shut down” all power consuming components, including itself for a predetermined amount of time, thus minimizing overall power consumption of the device. Sleep Mode may also be utilized within the other operating modes to selectively power down components that are not currently being utilized for measurements.

In Absolute Measurement Mode, the device would be capable of measuring the absolute values of color and clarity of present effluent, and compare such values against pre-determined, pre-set values that correspond to non-normal or anomalous values associated with the given effluent being analyzed.

In Trending Measurement Mode, the device would sample color and clarity data over a set period of time, thus establishing a temporal trend. In the case of increasing turbidity, the device may be programmed to activate any of the available alarms once a pre-set threshold for trending is exceeded. Trending of color changes over time may employ a similar mechanism.

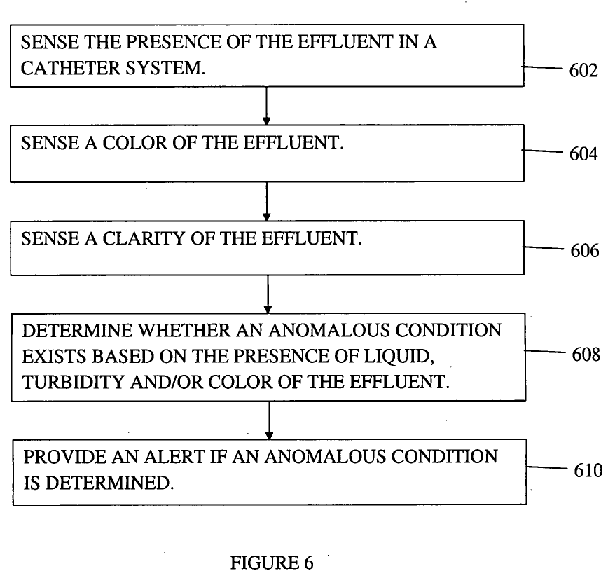

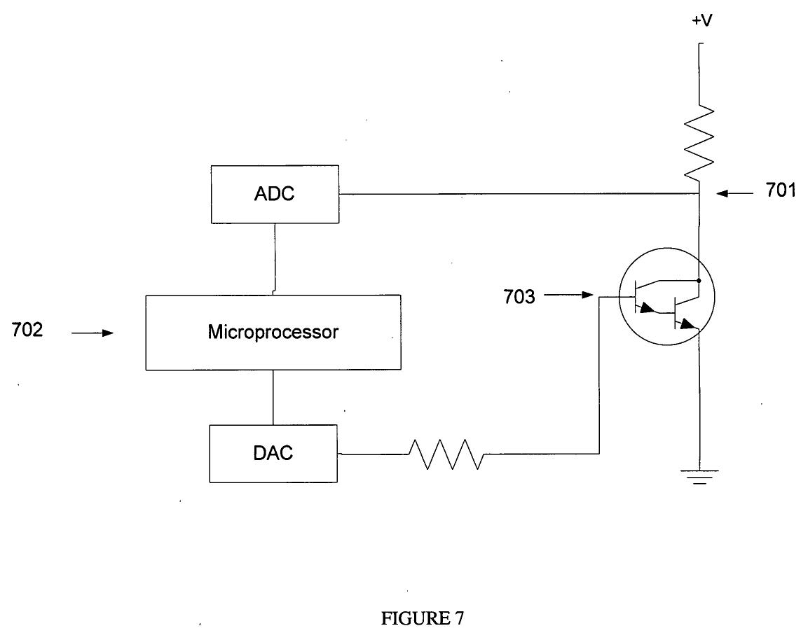

FIG. 6 illustrates a method for monitoring for anomalous conditions of an effluent of a catheter system. The presence of the effluent in a catheter system (e.g., tube, discharge bag, etc.) is sensed and/or monitored 602. Similarly, a color of the effluent is sensed or monitored 604. A clarity (or turbidity) of the effluent is also sensed, monitored, and/or determined 606. In one example, the clarity determination may be made using a photo-Darlington sensor or detector. Then, a determination may be made as to whether an anomalous condition exists based on the clarity and color of the effluent 608. If so, an alert (audio, visual, etc.) of the anomalous condition is provided 610.

The processing circuits or microprocessors described herein may be a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing components, e.g., a combination of a DSP and a microprocessor, a number of microprocessors, one or more microprocessors in conjunction with a DSP core, or any other such configuration.

The processing circuits/microprocessors, sensors and warning indicator(s) may be powered by a battery or an external power source. In various implementation, the monitoring device may include one or more sensors that operate together or independently to ascertain or detect one or more characteristics of liquid flowing through a (clear) discharge catheter tube or a discharge bag. In some implementations, the monitoring device may implement power management to reduce or minimize its power consumption, thereby extending the life of its power source (e.g., battery). For instance, the monitoring device may operate at a particular duty cycle in which the sensors and/or processing unit are only active for short periods of time. Additionally, the turbidity and color sensors may be operable only when the liquid sensor detects the presence of liquid.

One or more of the components and functions illustrated in FIGS. 1, 2, 3, 4, 5, 6, and/or 7 may be rearranged and/or combined into a single component or embodied in several components without departing from the invention. Additional elements or components may also be added without departing from the invention.

While certain exemplary embodiments have been described and shown in the accompanying drawings, it is to be understood that such embodiments are merely illustrative of and not restrictive on the broad invention, and that this invention is not limited to the specific constructions and arrangements shown and described, since various other modifications may occur to those ordinarily skilled in the art.

1. An apparatus for detecting anomalous conditions associated with effluents of catheter systems, comprising:
a refractive liquid sensor to sense the presence of the effluent in a catheter system;
a color sensor adapted to sense a color of the effluent; and
a clarity sensor adapted to sense a clarity of the effluent.

2. The apparatus of claim 1, wherein if the liquid sensor detects the presence of the effluent, color data collected by the color sensor and clarity data collected by the clarity sensor is considered valid, otherwise such data is ignored.

3. The apparatus of claim 1, wherein the liquid sensor includes a light source and a light sensor.

4. The apparatus of claim 1, wherein the liquid sensor detects a difference in displacement of the effluent by refraction when the effluent is present versus when it is absent.

5. The apparatus of claim 1, wherein the clarity sensor includes a sensitive light scattering detector to detect light scattered from a light source passing through the effluent at a right and oblique angle in order to determine the clarity of the effluent.

6. The apparatus of claim 5, wherein the dynamic range of the clarity sensor is between one (1) and five hundred (500) Nephelometric Turbidity Units (NTU) and a resolution sensitivity of at least five (5) NTU.

7. The apparatus of claim 6, wherein the clarity sensor includes a photo Darlington detector that is adapted to achieve a desired absolute signal measurement and a desired signal-to-noise ratio sufficient to achieve the dynamic range and resolution sensitivity.

8. The apparatus of claim 7, wherein the clarity sensor further includes a photo Darlington base drive feedback circuit to control the gain of the photo Darlington detector so as to eliminate non-linearity of gain associated with changes in ambient light and other noise sources.

9. The apparatus of claim 7, wherein the clarity sensor further includes a photo Darlington base drive feedback circuit to adjust absolute gain of the Darlington detector to provide greater overall dynamic range of the clarity sensor.

10. The apparatus of claim 7, wherein a low power laser diode is utilized as the light source.

11. The apparatus of claim 10, wherein the laser diode is positioned at a right and oblique angle to the photo Darlington detector so as to minimize light reflection noise within a test cavity and sense scattered light resulting from the presence of turbidity in the effluent.

12. The apparatus of claim 10, wherein a white light light-emitting diode is utilized to provide a transmitting light source for determining color of the catheter effluent.

13. The apparatus of claim 1, wherein the sensed color and clarity of the effluent are used to determine the occurrence of an anomalous condition.

14. The apparatus of claim 13, wherein a diffuser is utilized to provide a diffuse backlit light source for determining color of the catheter effluent.

15. The apparatus of claim 1, further comprising:
a microprocessor coupled to the liquid sensor, clarity sensor, and color sensor and adapted to collect effluent data and ascertain whether an anomalous condition is present.

16. The apparatus of claim 15, wherein the microprocessor is adapted to:
perform data trending analysis on collected effluent data, and trigger an alarm if the collected effluent data indicates a certain threshold has been exceeded.

17. The apparatus of claim 15, wherein the microprocessor is adapted to detect at least one of:
anomalous conditions associated with the effluent catheter systems,
early on-set of anomalous conditions, and
improvement or degradation of an already existing anomalous condition associated with catheter systems.

18. The apparatus of claim 15, wherein the microprocessor is adapted to perform statistical analysis of collected temporal sensor data to distinguish true alarm events from transient or noise events.

19. The apparatus of claim 15, wherein the microprocessor is adapted to:
measure elapsed time between liquid effluent detections by the liquid sensor, and trigger an alarm if a time threshold between liquid detections is exceeded to indicate un-timely effluent production.

20. The apparatus of claim 15, wherein the microprocessor is adapted to enter into a low power sleep mode when no effluent is sensed by the liquid sensor so as to enable a longer battery life.

21. The apparatus of claim 1, wherein the apparatus is attached to an effluent drain tube which is utilized as both an effluent flow path and a test cell for the sensors.

22. The apparatus of claim 21, wherein the apparatus is permanently attached to the effluent drain tube and is disposable.

23. The apparatus of claim 1, wherein the apparatus is attached to an effluent drain bag which is utilized as both an effluent reservoir and a test cell for the sensors.

24. An effluent monitoring device, comprising:
means for sensing the presence of the effluent in a catheter system;
means for sensing a color of the effluent; and
means for sensing a clarity of the effluent.

25. The effluent monitoring device of claim 24, further comprising:
means for determining whether an anomalous condition exists based on the clarity and color of the effluent; and
means for providing an alert of the anomalous condition.

26. The effluent monitoring device of claim 24, wherein if the presence of the effluent detected, color data for the effluent and clarity data for the effluent is considered valid, otherwise such data is ignored.

27. A method for monitoring for anomalous conditions of a effluent for a catheter system, comprising:
sensing the presence of the effluent in a catheter system;
sensing a color of the effluent; and
sensing a clarity of the effluent.

28. The method of claim 27, further comprising:
determining whether an anomalous condition exists based on the clarity and color of the effluent; and
providing an alert of the anomalous condition.
